(19) United States
(12) Patent Application Publication
(10) Pub. No.: US 2013/0338539 A1
(43) Pub. Date: Dec. 19, 2013

(54) SOFTWARE PROGRAM FOR MONITORING A HAND TREMOR OF AN END-USER VIA A COMPUTER MOUSE INPUT DEVICE

(71) Applicants: Gemma M. Bailey, Eastleigh (GB); Leon Chen, Bournemouth (GB); James K. Hook, Bristol (GB); Melanie S. Hopper, Winchester (GB); Susannah Marie Lindsay, Towcester (GB)

(72) Inventors: Gemma M. Bailey, Eastleigh (GB); Leon Chen, Bournemouth (GB); James K. Hook, Bristol (GB); Melanie S. Hopper, Winchester (GB); Susannah Marie Lindsay, Towcester (GB)

(73) Assignee: International Business Machines Corporation, Armonk, NY (US)

(21) Appl. No.: 13/523,097

(22) Filed: Jun. 14, 2012

(51) Int. Cl. A61B 5/11

(52) U.S. Cl. 600/595

(57) ABSTRACT

A computer receives movement information, wherein the movement information is sent by a computer mouse input device in response to an end-user moving the computer mouse input device. The computer determines a frequency at which an end-user typically tremors based on the movement information received during a specified time interval, by utilizing a fast Fourier transform operation. The computer determines current tremor frequencies of the end-user based on additional movement information, by utilizing the fast Fourier transform operation. The computer modifies a value assigned to a warning counter in response to at least one magnitude of one of the current tremor frequencies exceeding a magnitude of the frequency at which the end-user typically tremors. The computer sends an electronic notification in response to the value assigned to the warning counter exceeding a limit of an acceptable level of hand tremor.

### BACKGROUND

1. Field of the Invention

The present invention relates generally to monitoring a hand tremor of an end-user that is utilizing a computer, and more particularly to a tremor monitoring software program for monitoring the hand tremor of the end-user via a computer mouse input device connected to the computer.

2. Description of the Related Art

Utilizing a computer mouse input device requires a person to have a steady hand and a reasonable degree of manual dexterity. Thus, people with a hand tremor, may experience difficulty utilizing a computer mouse input device. Hand tremor is a series of small variable movements of a person's hand. Moreover, hand tremor can be a symptom associated with a variety of medical conditions that include diabetes, epilepsy, multiple sclerosis, Parkinson's disease, and Wilson's disease just to name a few examples.

To address the difficulty that an end-user with a hand tremor may experience when utilizing a computer mouse input device, existing technology primarily focuses on cancelling the effects of the hand tremor rather than helping the end-user take early preventative action to treat the hand tremor. Particularly, existing technology uses a variety of techniques that include comparing the velocity of an unintentional movement of a computer pointing device with a predetermined velocity threshold, wherein if the predetermined velocity threshold is exceeded then movement information generated by the computer pointing device, as a result of the unintentional movement, is discarded or attenuated in order to minimize the effects of the unintentional movement. In fact, end-users that are utilizing a computer and are experiencing a hand tremor may often rely solely on existing tremor cancelling technology to be able to effectively use the computer pointing device.

However, the magnitude of the end-user's hand tremor may increase over a period of time and become more difficult to cancel with the existing tremor cancelling technology. In addition, certain increases in the magnitude of the hand tremor may indicate that the end-user has a medical condition (e.g., diabetes or epilepsy) that is worsening and needs immediate attention of a licensed healthcare professional. If the end-user is alerted to certain increases in the magnitude of their hand tremor, then the end-user can take early preventative action to address the medical condition that they may have and thereby avoid having to rely solely on existing tremor cancelling technology to effectively use a computer pointing device.

### SUMMARY

Aspects of an embodiment of the present invention disclose a method, computer system, and program product for monitoring a hand tremor of an end-user. The computer receives movement information, wherein the movement information is sent by a computer mouse input device in response to an end-user moving the computer mouse input device. The computer determines a frequency at which an end-user typically tremors based on the movement information received during a specified time interval, by utilizing a fast Fourier transform operation. The computer determines current tremor frequencies of the end-user based on additional movement information, by utilizing the fast Fourier transform operation. The computer modifies a value assigned to a warning counter in response to at least one magnitude of one of the current tremor frequencies exceeding a tremor threshold parameter by a specified amount, wherein the tremor threshold parameter is equal to a magnitude of the frequency at which the end-user typically tremors. The computer sends an electronic notification in response to the value assigned to the warning counter exceeding a configurable warning level parameter that represents a limit of an acceptable level of hand tremor.

### BRIEF DESCRIPTION OF THE SEVERAL VIEWS OF THE DRAWINGS

The subject matter which is regarded as an embodiment of the present invention is particularly pointed out and distinctly claimed in the claims at the conclusion of the specification. One manner in which recited features of an embodiment of the present invention can be understood is by reference to the following detailed description of embodiments, taken in conjunction with the accompanying drawings in which:

FIG. 1 is a block diagram of a computer system having a computer utilizing a tremor monitoring software program to monitor a hand tremor of an end-user via a computer mouse input device connected to the computer according to an embodiment of the present invention.

FIGS. 2A-2C are flowcharts illustrating operations of the tremor monitoring software program according to an embodiment of the present invention.

FIG. 3 is a block diagram depicting internal and external components of the computer of FIG. 1 according to an embodiment of the present invention.

### DETAILED DESCRIPTION

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as JAVA, Smalltalk, C++ or the like, conventional procedural programming languages such as the “C” programming language, a hardware description language such as VERILOG, or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Exemplary embodiments now will be described more fully herein with reference to the accompanying drawings. This disclosure may, however, be embodied in many different forms and should not be construed as limited to the exemplary embodiments set forth herein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

Embodiments of the present invention provide a tremor monitoring software program, installed on a computer, for monitoring a hand tremor of an end-user via a computer mouse input device that is connected to the computer. Specifically, the tremor monitoring software program includes functionality for collecting movement information generated by the end-user's movement of the computer mouse input device. Tremor monitoring software program performs computations and analysis on the movement information that is collected, and can notify the end-user that they may have a medical condition based on the computations and analysis.

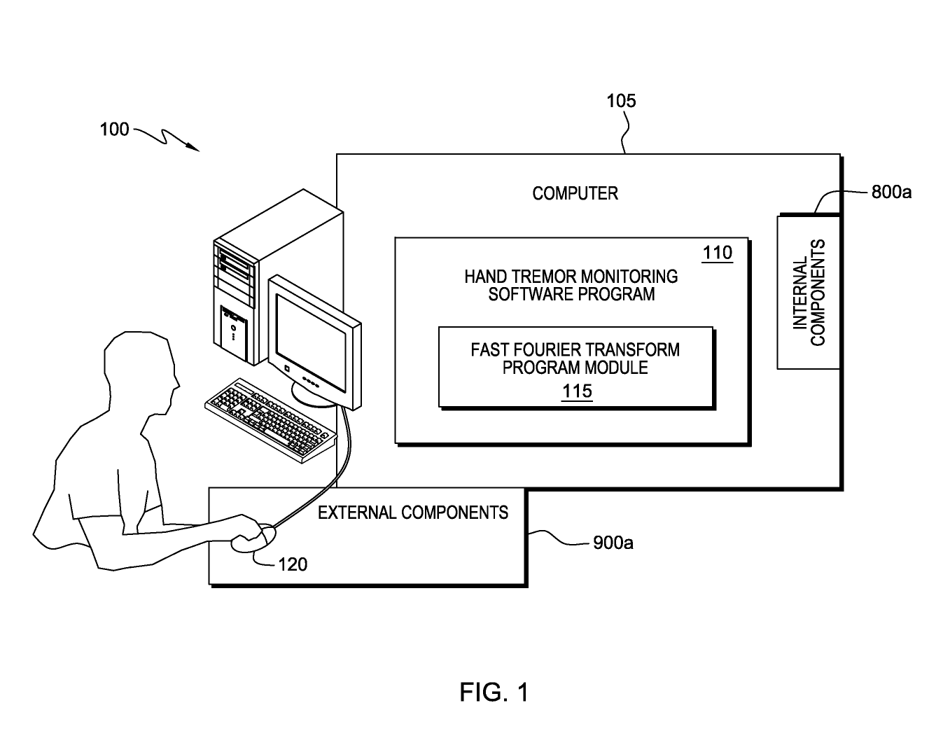

FIG. 1 illustrates computer system 100 that includes computer 105 having hand tremor monitoring software program 110 installed. Tremor monitoring software program 110 includes fast Fourier transform program module 115. In addition, computer 105 includes internal components 800a and external components 900a. External components 900a include a variety of peripheral devices such as computer mouse input device 120 that is connected to computer 105.

In the disclosed embodiment, tremor monitoring software program 110 is configured to interact with computer mouse input device 120 and receive movement information. Computer mouse input device 120 can be a computer mouse or other type of computer input device that is capable of moving a cursor. The movement information is sent by computer mouse input device 120 to computer 105, in response to a particular end-user utilizing their hand to move the computer mouse input device. The movement information is one or more signals from which data representing a direction, a distance, and a timestamp of the particular end-user's movement of computer mouse input device 120 can be determined. Distance is the antiderivative of velocity with respect to time, and velocity is the antiderivative of acceleration with respect to time. Therefore, in one embodiment, tremor monitoring software program 110 can compute velocity and acceleration, merely based on data representing the distance and the timestamp of the particular end-user's movement. Thus, tremor monitoring software program 110 can track a cursor that corresponds with computer mouse input device 120, and determine a position, velocity, or acceleration of the cursor over time. In still other embodiments, the movement information may also be obtained by connecting an accelerometer to computer mouse input device 120 and wirelessly transmitting the movement information, via a COM port, to tremor monitoring software program 110.

Furthermore, based on the movement information received by tremor monitoring software program 110 during a specified time interval, the tremor monitoring software program can determine a base tremor frequency of the particular end-user's hand tremor. The base tremor frequency is the frequency at which the hand of the particular end-user typically tremors. Specifically, the base tremor can be hand movements in a somewhat rhythmic fashion, and thus the base tremor frequency for a particular end-user can be defined as an expected amount of hand movements of the end-user per unit of time. For example, if the particular end-user is moving computer mouse input device 120 in a linear movement (e.g., from one side of the screen to the other), then non-tremor hand movement can be represented by a straight line on a graph illustrating distance, velocity, or acceleration versus time. The straight line can have a constant gradient, wherein the gradient can be a zero value for velocity or acceleration. Thus, hand movement affected by tremor can fluctuate above and below the straight line. The frequency of the fluctuation can represent the frequency at which the hand of the particular end-user typically tremors (i.e., the base tremor frequency). In the disclosed embodiment, the specified time interval has a duration of about 30 minutes, and the time interval first begins to run in response to the first movement of computer mouse input device 120 from a stationary position. In alternative embodiments, the specified time interval can be configured by a system programmer/administrator to be greater than about 30 minutes to allow enough time for tremor monitoring software program 110 to receive a suitable amount of the movement information in order to determine the base tremor frequency with a higher level of accuracy.

More specifically, to determine the base tremor frequency, tremor monitoring software program 110 performs a fast Fourier transform (FFT) operation on the movement information that is received during the specified time interval. Particularly, in the disclosed embodiment FFT functionality for performing the FFT operation is provided by fast Fourier transform program module 115 of tremor monitoring software program 110. FFT program module 115 receives the movement information sent from computer mouse input device 120 and utilizes the FFT operation to decompose the movement information into values that represent frequencies of the particular end-user's hand tremor during the specified time interval. Particularly, FFT program module 115 can decompose a continuous signal representing movement information into a sum of sine and cosine waves with frequencies in 2π multiples. Each of the waves have coefficient values corresponding to the given signal. The coefficient values represent the magnitudes of frequencies of the particular end-user's hand tremor, and the magnitudes can then be analyzed to see if they increase or decrease over time. In one embodiment, tremor monitoring software program 110 may provide FFT functionality by utilizing program functions (e.g., fft(data) of programming language PYTHON) from one or more program libraries, wherein the program libraries contain program code that can be used by tremor monitoring software program 110 in a modular programming approach.

Subsequent to the FFT program module 115 decomposing the movement information into the values that represent frequencies of the particular end-user's hand tremor during the specified time interval, tremor monitoring software program 110 iterates over the values, and performs comparisons between each of the values to identify a commonality among the values (i.e., the tremor frequencies at which coefficient values have remained nearly constant returned by FFT program module 115) and determine the base tremor frequency. Particularly, the commonality can be identified by analyzing coefficients returned by the FFT program module 115, wherein the coefficients represent magnitudes of frequencies of the particular end-user's hand tremor. Next, tremor monitoring software program 110 determines the magnitude of the base tremor frequency based on the commonality and sets a tremor threshold parameter to be the magnitude of the base tremor frequency. After the tremor threshold parameter is set, tremor monitoring software program 110 continues to monitor computer mouse input device 120, every 30 seconds, and receive additional movement information based on the particular end-user's subsequent movements of the computer mouse input device. Tremor monitoring software program 110 uses the additional movement information received, if any, and the FFT operation to determine current tremor frequencies of the particular end-user's hand tremor. The current tremor frequencies are frequencies at which the hand of the particular end-user tremors, subsequent to the tremor threshold parameter being set.

Specifically, the current tremor frequencies of the particular end-user's hand tremor are determined by performing the FFT operation on the additional movement information using the FFT functionality of FFT program module 115, wherein the FFT program module receives the additional movement information sent from computer mouse input device 120 and decomposes the additional movement information into values that represent the current tremor frequencies of the particular end-user's hand tremor. Next, tremor monitoring software program 110 computes a magnitude of each of the current tremor frequencies using the same method that was used to compute the magnitude of the base tremor frequency, and compares the magnitude of the current tremor frequency equal to the base tremor frequency, to the tremor threshold parameter that was previously set. Particularly, the magnitude of the current tremor frequency can be computed by utilizing coefficients returned by the FFT program module 115, wherein the coefficients correspond to magnitudes associated with one or more frequencies of the particular end-user's hand tremor. In the disclosed embodiment, if the magnitude of the current tremor frequency exceeds the tremor threshold parameter by a specified amount, then a value assigned to a warning counter is incremented by 1. The specified amount is configurable based on the particular end-user's needs.

Moreover, if the value assigned to the warning counter exceeds a configurable warning level parameter, then tremor monitoring software program 110 sends an electronic notification to the particular end-user. The warning level parameter represents a limit of an acceptable level of hand tremor. In other embodiments, tremor monitoring software program 110 can send an electronic notification to both the particular end-user and one or more designated recipients based on preferences of the particular end-user. The particular end-user can select the preferences for the electronic notification within a graphical user interface (GUI) of tremor monitoring software program 110. Subsequent to sending the electronic notification, if at least more than fifty percent of current tremor frequencies of the particular end-user's hand tremor have a magnitude below the tremor threshold parameter, then tremor monitoring software program 110 resets the value assigned to the warning counter to null.

Tremor monitoring software program 110 can operate in at least two different modes. One mode is referred to as degradation mode, wherein the base tremor frequency and tremor threshold parameter are updated on a daily basis or less frequently than a daily basis. The purpose of the degradation mode is to identify gradual deterioration in the particular end-user's tremor condition over an extended period of time (e.g., over a 24 hour time period or greater). The other mode is referred to as emergency mode, wherein the base tremor frequency and tremor threshold parameter are updated at least more frequently than a daily basis. Thus, the purpose of the emergency mode is to identify abrupt increases in the particular end-user's tremor condition over a short period of time (e.g., over an hour period, a half-hour period, or less than a half-hour period).

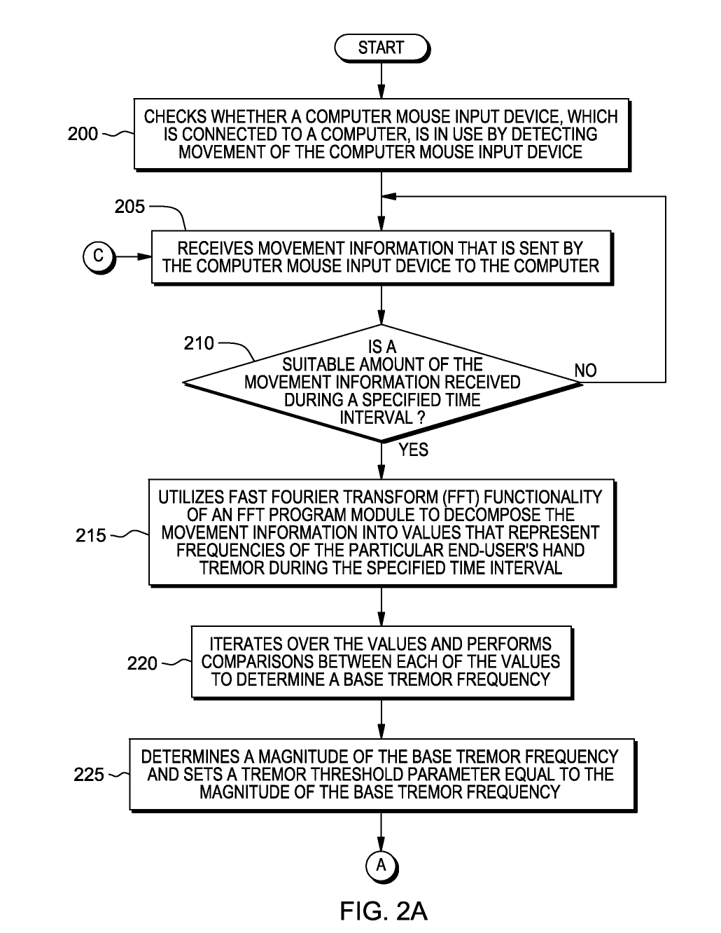

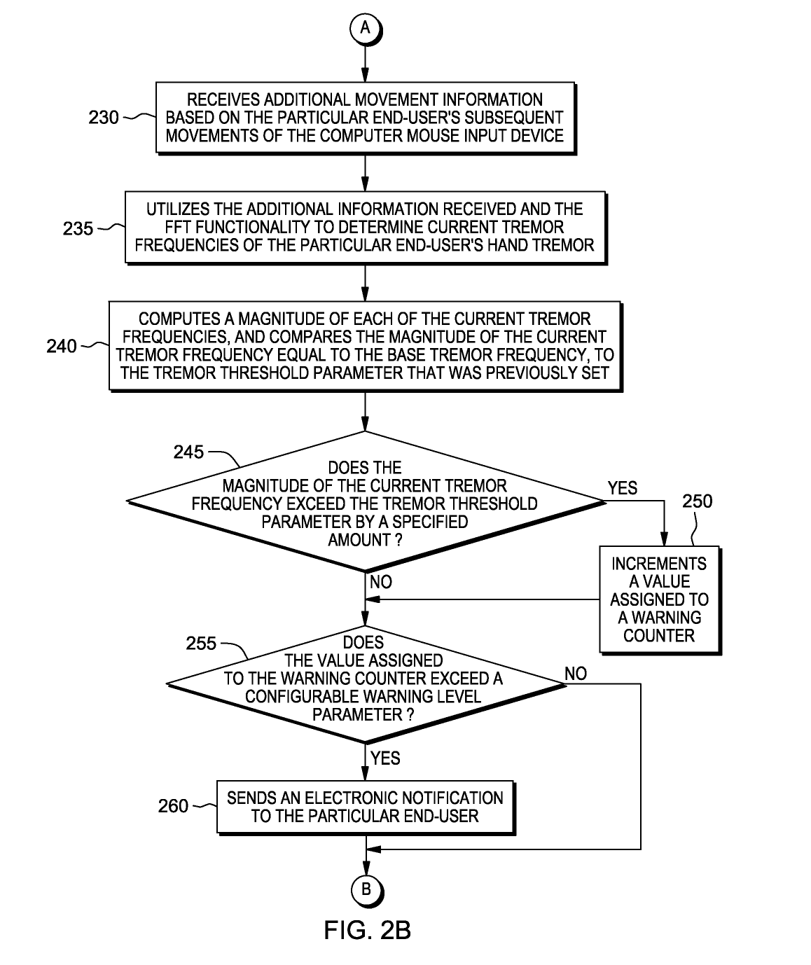

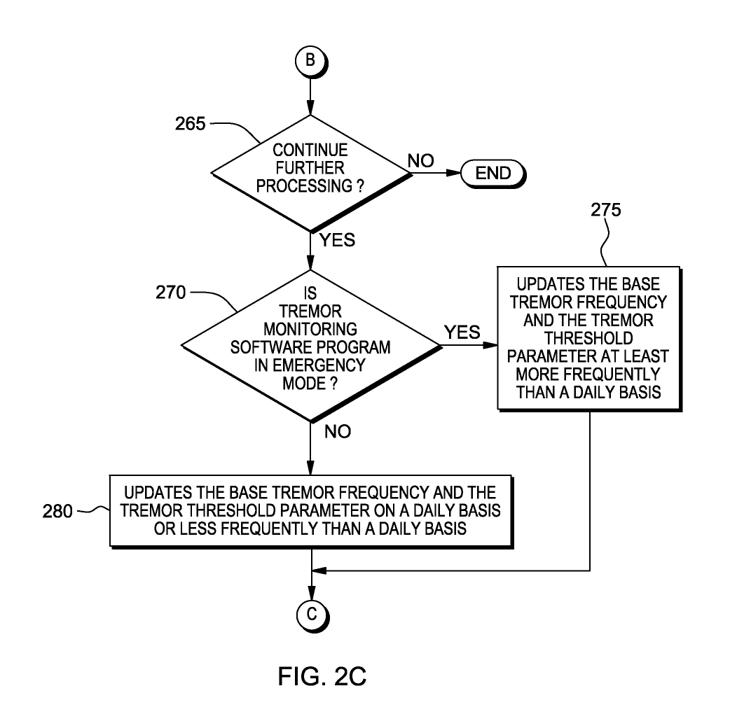

FIGS. 2A-2C are flowcharts illustrating the steps of hand tremor monitoring software program 110 monitoring a hand tremor of a particular end-user utilizing computer mouse input device 120. In one embodiment, tremor monitoring software program 110 checks whether computer mouse input device 120, which is connected to computer 105, is in use by detecting movement of the computer mouse input device (block 200). Specifically, in response to a particular end-user's hand moving computer mouse input device 120, the computer mouse input device sends movement information to computer 105. The movement information includes data from which a direction, a distance, and a timestamp of the particular end-user's movement of computer mouse input device 120 can be determined.

Next, in the disclosed embodiment, tremor monitoring software program 110 receives movement information that is sent by computer mouse input device 120 to computer 105 (block 205). If a suitable amount of the movement information is received during a specified time interval (the “YES” branch of decision block 210), then tremor monitoring software program 110 utilizes FFT functionality of FFT program module 115 to decompose the movement information into values that represent frequencies of the particular end-user's hand tremor during the specified time interval (block 215). Otherwise, if a suitable amount of the movement information has not yet been received during the specified time interval (the “NO” branch of decision block 210) then during the specified time interval, tremor monitoring software program 110 continues to receive the movement information from the computer mouse input device 120. A suitable amount of the movement information is needed in order to determine a base tremor frequency at which the hand of the particular end-user typically tremors, with a high level of accuracy. Moreover, a suitable amount of movement information is received during the specified time interval if subsequent to receipt of movement information, FFT program module 115 is able to decompose a sufficient amount of values (e.g., at least about 60 values) that represent frequencies of the particular end-user's tremor. In the disclosed embodiment, the specified time interval has a duration of about 30 minutes, and the time interval first begins to run in response to a first movement of computer mouse input device 120 from a stationary position. However, the specified time interval can be configured by a system programmer/administrator to allow more than 30 minutes for tremor monitoring software program 110 to receive a suitable amount of the movement information.

Furthermore, subsequent to utilizing FFT program module 115, tremor monitoring software program 110 iterates over the values that represent the frequencies of the particular end-user's hand tremor during the specified time interval, and performs comparisons between each of the values to determine a base tremor frequency (block 220). Next, tremor monitoring software program 110 determines a magnitude of the base tremor frequency and sets a tremor threshold parameter equal to the magnitude of the base tremor frequency (block 225).

After the tremor threshold parameter is set, tremor monitoring software program 110 continues to monitor computer mouse input device 120, every 30 seconds, and receive additional movement information based on the particular end-user's subsequent movements of the computer mouse input device (block 230). Tremor monitoring software program 110 utilizes the additional movement information received and the FFT functionality to determine current tremor frequencies of the particular end-user's hand tremor (block 235).

Next, tremor monitoring software program 110 computes a magnitude of each of the current tremor frequencies, and compares the magnitude of the current tremor frequency equal to the base tremor frequency, to the tremor threshold parameter that was previously set (block 240). If the magnitude of the current tremor frequency equal to the base tremor frequency exceeds the tremor threshold parameter by a specified amount (the “YES” branch of decision block 245), then tremor monitoring software program 110 increments a value assigned to a warning counter (block 250).

Subsequent to incrementing the value assigned to the warning counter, or if not at least one magnitude of one of the current tremor frequencies exceed the tremor threshold parameter by the specified amount (the “NO” branch of decision block 245), then tremor monitoring software program 110 determines whether the value assigned to the warning counter exceeds a configurable warning level parameter (decision block 255). If the value assigned to the warning counter does not exceed the configurable warning level parameter (the “NO” branch of decision block 255), tremor monitoring software program 110 determines whether to continue further processing (decision block 265). However, if the value assigned to the warning counter exceeds the configurable warning level parameter (the “YES” branch of decision block 255), then tremor monitoring software program 110 sends an electronic notification to the particular end-user (block 260). The electronic notification can alert the particular end-user that their hand tremor has increased beyond a limit of an acceptable level of hand tremor. As mentioned above, the particular user can designate recipients to receive the electronic notification by selecting preferences for the electronic notification within a graphical user interface (GUI) of tremor monitoring software program 110.

Next, after the electronic notification is sent to the particular end-user and/or designated recipients, or after tremor monitoring program 110 determines that the value assigned to the warning counter does not exceed the configurable warning level parameter and if the tremor monitoring software program determines not to continue further processing (the “NO” branch of decision block 265), then the tremor monitoring software program ends. Otherwise, if tremor monitoring software program 110 determines to continue further processing (the “YES” branch of decision block 265), and tremor monitoring software program 110 is in emergency mode (the “YES” branch of decision block 270), then the tremor monitoring software program updates the base tremor frequency and tremor threshold parameter at least more frequently than a daily basis (block 275). Otherwise, if tremor monitoring software program 110 is not in emergency mode (the “NO” branch of decision block 270), then the tremor monitoring software program is in degradation mode and updates the base tremor frequency and tremor threshold parameter on a daily basis or less frequently than a daily basis (block 280). Next, tremor monitoring software program 110 continues to receive movement information sent by the computer mouse input device 120 (block 205). Subsequently, during further processing and if an electronic notification has been sent, tremor monitoring software program 110 may reset the value assigned to the warning counter to null if at least more than fifty percent of current tremor frequencies have a magnitude that is below the tremor threshold parameter.

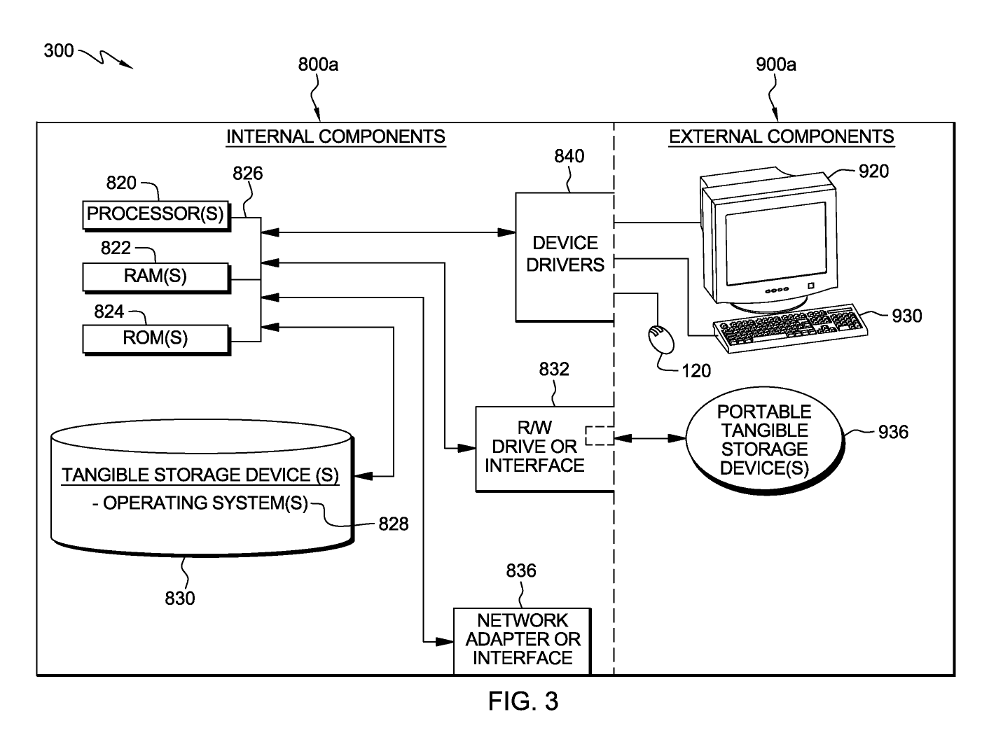

FIG. 3 is block diagram 300 depicting a set of internal components 800a and a set of external components 900a that correspond to computer 105. Internal components 800a includes one or more processors 820, one or more computer-readable RAMs 822, one or more computer-readable ROMs 824 on one or more buses 826, and one or more operating systems 828 and one or more computer-readable tangible storage devices 830. The one or more operating systems 828 and hand tremor monitoring software program 110 having FFT program module 115 are stored on one or more of the respective computer-readable tangible storage devices 830 for execution by one or more of the respective processors 820 via one or more of the respective RAMs 822 (which typically include cache memory). In the embodiment illustrated in FIG. 3, each of the computer-readable tangible storage devices 830 is a magnetic disk storage device of an internal hard drive. Alternatively, each of the computer-readable tangible storage devices 830 is a semiconductor storage device such as ROM 824, EPROM, flash memory or any other computer-readable tangible storage device that can store a computer program and digital information.

The set of internal components 800a includes a R/W drive or interface 832 to read from and write to one or more portable computer-readable tangible storage devices 936 such as CD-ROM, DVD, memory stick, magnetic tape, magnetic disk, optical disk or semiconductor storage device. Tremor monitoring software program 110 having FFT program module 115 can be stored on one or more of the respective portable computer-readable tangible storage devices 936, read via the respective R/W drive or interface 832 and loaded into the respective hard drive 830.

Furthermore, each set of internal components 800a and 800b also includes a network adapter or interface 836 such as TCP/IP adapter card, wireless wi-fi interface card, or 3G or 4G wireless interface card or other wired or wireless communication link. Tremor monitoring software program 110 having FFT program module 115 can be downloaded to respective computer 105 from an external computer or external storage device via a network (for example, the Internet, a LAN, or a WAN) and respective network adapters or interfaces 836. From the network adapter or interface 836, tremor monitoring software program 110 having FFT program module 115 is loaded into respective hard drive 830. The network may comprise copper wires, optical fibers, wireless transmission, routers, firewalls, switches, gateway computers and/or servers.

The set of external components 900a can include a computer display monitor 920, a keyboard 930, and a computer mouse input device 120. External components 900a can also include touch screens, virtual keyboards, touch pads, pointing devices, and other human interface devices. The set of internal components 800a also includes device drivers 840 to interface to computer display monitor 920, keyboard 930 and computer mouse input device 120. The device drivers 840, R/W drive or interface 832 and network adapter or interface 836 comprise hardware and software in which the software is stored in computer-readable tangible storage device 830 and/or ROM 824.

It should be appreciated that FIG. 3 provides only an illustration of one implementation and does not imply any limitations with regard to the environments in which different embodiments may be implemented. A variety of modifications to the depicted environments may be implemented. Moreover, a variety of modifications to the depicted environments may be made based on design and implementation requirements.

In accordance with the foregoing, a method, computer system, and computer program product have been disclosed for monitoring a hand tremor of an end-user via a computer mouse input device that is connected to a computer. However, numerous modifications substitutions can be made without deviating from the scope of an embodiment of the invention. Therefore, one or more embodiments of the invention have been disclosed by way of example and not limitation.

What is claimed is:

1. A method for monitoring a hand tremor of an end-user, the method comprising the steps of:
a computer receiving movement information, wherein the movement information is sent by a computer mouse input device in response to an end-user moving the computer mouse input device;
the computer determining a frequency at which an end-user typically tremors based on the movement information received during a specified time interval, by utilizing a fast Fourier transform operation;
the computer determining current tremor frequencies of the end-user based on additional movement information, by utilizing the fast Fourier transform operation;
the computer modifying a value assigned to a warning counter in response to at least one magnitude of one of the current tremor frequencies exceeding a tremor threshold parameter by a specified amount, wherein the tremor threshold parameter is equal to a magnitude of the frequency at which the end-user typically tremors; and
the computer sending an electronic notification in response to the value assigned to the warning counter exceeding a configurable warning level parameter that represents a limit of an acceptable level of hand tremor.

2. The method of claim 1, wherein the step of receiving movement information comprises receiving one or more signals that include data from which a direction, a distance, and a timestamp of the end-user's movement of the computer mouse input device is determined.

3. The method of claim 1, wherein the step of the computer determining the frequency at which the end-user typically tremors based on the movement information received during the specified time interval, by utilizing the fast Fourier transform comprises the steps of:
decomposing the movement information into values that represent frequencies of the hand tremor of the end-user during the specified time interval; and
computing the magnitude of the frequency at which the end-user typically tremors by utilizing coefficients resulting from the fast Fourier transform operation.

4. The method of claim 1, wherein the frequency at which the end-user typically tremors and tremor threshold parameter are updated on a daily basis or less frequently than a daily basis.

5. The method of claim 1, wherein the frequency at which the end-user typically tremors and tremor threshold parameter are updated at least more frequently than a daily basis.

6. The method of claim 1, wherein the step of the computer determining the current tremor frequencies of the end-user based on additional movement information, by utilizing the fast Fourier transform comprises the steps of:
decomposing the additional movement information into values that represent the current tremor frequencies; and
computing the magnitude of each of the current tremor frequencies by utilizing coefficients resulting from the fast Fourier transform operation.

7. The method of claim 1, wherein the step of the computer modifying the value assigned to the warning counter comprises:
increasing the value assigned to the warning counter by 1 if at least one magnitude of one of the current tremor frequencies exceed the tremor threshold parameter by a specified amount; and
resetting the value assigned to the warning counter to null if at least more than fifty percent of the current tremor frequencies have a magnitude below the tremor threshold parameter.

8. A computer program product for monitoring a hand tremor of an end-user comprising:
a computer readable storage medium and program instructions stored on the computer readable storage medium, the program instructions comprising:
program instructions to receive movement information, wherein the movement information is sent by a computer mouse input device, which is connected to the computer, in response to an end-user moving the computer mouse input device;
program instructions to determine a frequency at which an end-user typically tremors based on the movement information received during a specified time interval, by utilizing a fast Fourier transform operation;
program instructions to determine current tremor frequencies of the end-user based on additional movement information, by utilizing the fast Fourier transform operation;
program instructions to modify a value assigned to a warning counter in response to at least one magnitude of one of the current tremor frequencies exceeding a tremor threshold parameter by a specified amount, wherein the tremor threshold parameter is equal to a magnitude of the frequency at which the end-user typically tremors; and
program instructions to send an electronic notification in response to the value assigned to the warning counter exceeding a configurable warning level parameter that represents a limit of an acceptable level of hand tremor.

9. The computer program product of claim 8, wherein the step of receiving movement information comprises receiving one or more signals that include data from which a direction, a distance, and a timestamp of the end-user's movement of the computer mouse input device is determined.

10. The computer program product of claim 8, wherein the program instructions to determine the frequency at which the end-user typically tremors based on the movement information received during the specified time interval, by utilizing the fast Fourier transform comprises the steps of:
decomposing the movement information into values that represent frequencies of the hand tremor of the end-user during the specified time interval; and
computing the magnitude of the frequency at which the end-user typically tremors by utilizing coefficients resulting from the fast Fourier transform operation.

11. The computer program product of claim 8, wherein the frequency at which the end-user typically tremors and tremor threshold parameter are updated on a daily basis or less frequently than a daily basis.

12. The computer program product of claim 8, wherein the frequency at which the end-user typically tremors and tremor threshold parameter are updated at least more frequently than a daily basis.

13. The computer program product of claim 8, wherein the program instructions to determine the current tremor frequencies of the end-user based on additional movement information, by utilizing the fast Fourier transform comprises the steps of:
decomposing the additional movement information into values that represent the current tremor frequencies; and
computing the magnitude of each of the current tremor frequencies by utilizing coefficients resulting from the fast Fourier transform operation.

14. The computer program product of claim 8, wherein the program instructions to modify the value assigned to the warning counter comprises:
increasing the value assigned to the warning counter by 1 if at least one magnitude of one of the current tremor frequencies exceed the tremor threshold parameter by a specified amount; and
resetting the value assigned to the warning counter to null if at least more than fifty percent of the current tremor frequencies have a magnitude below the tremor threshold parameter.

15. A computer system for monitoring a hand tremor of an end-user comprising:
one or more processors, one or more computer readable memories, one or more computer readable storage media, and program instructions stored on the one or more storage media for execution by the one or more processors via the one or more memories, the program instructions comprising:
program instructions to receive movement information, wherein the movement information is sent by a computer mouse input device, which is connected to the computer, in response to an end-user moving the computer mouse input device;
program instructions to determine a frequency at which an end-user typically tremors based on the movement information received during a specified time interval, by utilizing a fast Fourier transform operation;
program instructions to determine current tremor frequencies of the end-user based on additional movement information, by utilizing the fast Fourier transform operation;
program instructions to modify a value assigned to a warning counter in response to at least one magnitude of one of the current tremor frequencies exceeding a tremor threshold parameter by a specified amount, wherein the tremor threshold parameter is equal to a magnitude of the frequency at which the end-user typically tremors; and
program instructions to send an electronic notification in response to the value assigned to the warning counter exceeding a configurable warning level parameter that represents a limit of an acceptable level of hand tremor.

16. The computer system of claim 15, wherein the step of receiving movement information comprises receiving one or more signals that include data from which a direction, a distance, and a timestamp of the end-user's movement of the computer mouse input device is determined.

17. The computer system of claim 15, wherein the program instructions to determine the frequency at which the end-user typically tremors based on the movement information received during the specified time interval, by utilizing the fast Fourier transform comprises the steps of:
decomposing the movement information into values that represent frequencies of the hand tremor of the end-user during the specified time interval; and
computing the magnitude of the frequency at which the end-user typically tremors by utilizing coefficients resulting from the fast Fourier transform operation.

18. The computer system of claim 15, wherein the frequency at which the end-user typically tremors and tremor threshold parameter are updated on a daily basis or less frequently than a daily basis.

19. The computer system of claim 15, wherein the frequency at which the end-user typically tremors and tremor threshold parameter are updated at least more frequently than a daily basis.

20. The computer system of claim 15, wherein the program instructions to determine the current tremor frequencies of the end-user based on additional movement information, by utilizing the fast Fourier transform comprises the steps of:
decomposing the additional movement information into values that represent the current tremor
frequencies; and
computing the magnitude of each of the current tremor frequencies by utilizing coefficients resulting from the fast Fourier transform operation.

21. The computer system of claim 15, wherein the program instructions to modify the value assigned to the warning counter comprises:
increasing the value assigned to the warning counter by 1 if at least one magnitude of one of the current tremor frequencies exceed the tremor threshold parameter by a specified amount; and
resetting the value assigned to the warning counter to null if at least more than fifty percent of the current tremor frequencies have a magnitude below the tremor threshold parameter.
